(19) United States
(12) Patent Application Publication
(10) Pub. No.: US 2004/0092531 A1
(43) Pub. Date: May 13, 2004

(54) Active substance combination containing an opioid having a fentanyl-type structure and ketamine

(76) Inventors: Boris Chizh, Cambridge (GB); Thomas Christoph, Aachen (DE); Werner Englberger, Stolberg (DE)

Correspondence Address: PERMAN & GREEN, 425 POST ROAD, FAIRFIELD, CT, 06824, US

(21) Appl. No.: 10/296,098

(22) PCT Filed: May 10, 2001
(86) PCT No.: PCT/EP01/05348

(30) Foreign Application Priority Data
May 26, 2000 (DE) 10025946.4

(51) Int. Cl. A61K 31/497; A61K 31/453; A61K 31/445; A61K 31/155

(52) U.S. Cl. 514/255.05; 514/317; 514/326; 514/632

(57) ABSTRACT

The invention relates to an active substance combination that contains as the active substance component a) at least one opioid compound that has a fentanyl-type structure and/or the enantiomers and/or the diastereomers thereof and/or at least one corresponding pharmaceutically acceptable salt, and as the active substance component b) ketamine and/or at least one of its physiologically acceptable salts. The weight ratio of active substance component a) to active substance component b) ranges from 1:20 to 1:1500. The invention also relates to medicament formulations and medicaments that contain the inventive active substance combination and to the use of said active substance combination for producing medicaments.

The invention relates to a combination of active ingredients containing the following active components: a) at least one opioid compound with a fentanyl-type structure and/or the enantiomers and/or the diastereomers thereof and/or at least one corresponding pharmaceutically tolerable salt and b) ketamine and/or at least one of its physiologically tolerable salts, the weight ratio of active component a) to active component b) being in the range of 1:20 to 1:1500. It also relates to medicament formulations and medicaments containing this combination of active ingredients and to the use of this combination of active ingredients for the manufacture of medicaments.

Pain is one of the basic symptoms of clinical practice and there is a worldwide requirement for effective pain therapies. The urgent need for a patient-friendly and specific treatment for chronic and non-chronic pain conditions, i.e. successful and satisfactory pain treatment for patients, is documented in the large number of scientific studies which have appeared recently in the field of applied analgesics and fundamental research into nociception.

Neuropathic pain represents a particular form of chronic pain, which is produced by various injuries to the peripheral or central nervous system and which can only be treated inadequately with traditional analgesics such as opioids, for example. Opioids also have the disadvantage that they often have only a very short duration of effect and very often have undesirable side effects such as respiratory depression, nausea, vomiting, dependency, sedation, constipation or the development of tolerance, for example.

One class of analgesically effective compounds suitable for controlling neuropathic pain is the N-methyl-D-aspartate (NMDA) antagonists. However, these frequently have only a very short duration of effect and often exhibit very marked undesirable side effects such as hallucinogenic effects, impaired coordination, sedation, nausea or itching, for example.

U.S. Pat. No. 5,321,012 discloses pharmaceutical compositions comprising a narcotic analgesic and another active ingredient such as an NMDA antagonist, for example. The combined administration of the narcotic analgesic with such active ingredients should prevent the development of tolerance or the development of dependency on the narcotic analgesic.

In Anesth. Analg., 1998, 86, pp 1250 et seq, an analgesic combination comprising ketamine and alfentanil is disclosed in which the two active ingredients are present in a weight ratio of 10 to 1. No synergistic interaction was able to be found for this combination of active ingredients.

The requirement has therefore arisen for medicaments with lasting effect for controlling pain, especially for controlling neuropathic pain, exhibiting as few of the side effects of the opioid analgesics as possible, such as respiratory depression, nausea, vomiting, dependency, sedation, constipation or the development of tolerance, and as few of the side effects of the NMDA antagonists such as hallucinogenic effects, impaired coordination or itching, for example.

Surprisingly, it has now been found that a combination of active ingredients containing the following active components: a) at least one opioid compound with a fentanyl-type structure and/or the enantiomers and/or the diastereomers thereof and/or at least one corresponding physiologically tolerable salt and b) ketamine and/or at least one of its physiologically tolerable salts in a certain weight ratio exhibits a lasting analgesic effect and is therefore suitable for controlling pain, especially for controlling neuropathic pain.

The object of the invention is thus a combination of active ingredients containing:

a) at least one opioid compound with a fentanyl-type structure and/or the enantiomers and/or the diastereomers thereof and/or at least one corresponding physiologically tolerable salt and

b) ketamine and/or at least one of its physiologically tolerable salts,

the weight ratio of active component a) to active component b) being in the range of 1:20 to 1:1500.

The manufacture of opioid compounds with a fentanyl-type structure, the enantiomers, the diastereomers thereof and the corresponding physiologically tolerable salts is known from the literature, e.g. E. Friderichs, T. Christoph, H. Buschmann, “Analgesics and Antipyretics”, Ullmann's Encyclopaedia of Industrial Chemistry, Sixth Edition on CD-ROM, Wiley-VCH, Weinheim, 2000. The manufacture of ketamine is also known from the literature, e.g. BE-PS-634 208, U.S. Pat. No. 3,254,124 and C. L. Stevens et al, J. Org. Chem. 30, p 2967, 1965. The corresponding disclosures are attached as references.

The combination of active ingredients according to the invention surprisingly shows a lasting analgesic effect, far exceeding the duration of effect of either of the two active ingredients alone, and is therefore outstandingly suitable for controlling pain, especially for controlling neuropathic and/or acute pain, the undesirable side effects which usually occur with the administration of opioids or NMDA antagonists do not occur or only occur for a considerably shorter period of time and only in a clearly less severe form than with the administration of the active components singly.

The combination of active ingredients according to the invention may contain opioid compounds with a fentanyl-type structure, the diastereomers thereof, the enantiomers thereof and the corresponding physiologically tolerable salts thereof singly or in mixtures of at least two of these compounds. The combination of active ingredients according to the invention preferably contains one opioid compound with a fentanyl-type structure, the enantiomers thereof, the diastereomers thereof or a corresponding physiologically tolerable salt.

In a preferred embodiment of the invention, the combination of active ingredients according to the invention contains at least one compound of general formula I as component a)

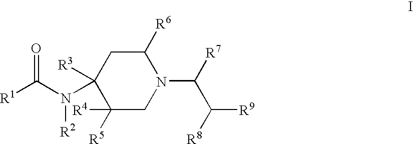

where

group R¹ stands for a C₁₋₃-alkyl, a C₁₋₃-alkoxymethyl or a 2-furanyl group,

group R² stands for a phenyl group or a phenyl group optionally substituted with fluorine in the ortho-position or a 2-pyrazinyl group,

group R³ stands for H, a C₁₋₃-alkoxymethyl, a C₁₋₃-alkoxycarbonyl or a phenyl group,

groups R⁴ and R⁵, the same or different, each stand for H, OH or a C₁₋₃-alkyl group,

groups R⁶ and R⁷, the same or different, each stand for H or a C₁₋₃-alkyl group,

group R⁸ stands for H or OH and

group R⁹ stands for a phenyl, a 2-thienyl, a C₁₋₃-alkoxycarbonyl or a 1-ethyl-1,4-dihydro-tetrazol-5-one group,

and/or one of the enantiomers thereof and/or one of the diastereomers thereof and/or at least one corresponding physiologically tolerable salt.

The combination of active ingredients according to the invention particularly preferably contains fentanyl, alfentanil, brifentanil, carfentanil, fenaridine, fentatienil, lofentanil, ocfentanil, mefentanil, mirfentanil, remifentanil, sufentanil, trefentanil and/or one of the enantiomers thereof and/or one of the diastereomers thereof and/or at least one corresponding physiologically tolerable salt or a mixture of at least two of the above mentioned compounds.

A physiologically tolerable salt of the opioid compound with a fentanyl-type structure and/or enantiomers thereof and/or diastereomers thereof may preferably be hydrochloride, hydrobromide, sulphate, sulphonate, phosphate, tartrate, embonate, formiate, acetate, propionate, benzoate, oxalate, succinate, citrate, glutamate, fumarate, aspartate, glutarate, stearate, butyrate, malonate, lactate, mesylate or a mixture of at least two of these salts.

A physiologically tolerable salt of the ketamine may preferably be hydrochloride, hydrobromide, sulphate, sulphonate, phosphate, tartrate, embonate, formiate, acetate, propionate, benzoate, oxalate, succinate, citrate, glutamate, fumarate, aspartate, glutarate, stearate, butyrate, malonate, lactate, mesylate or a mixture of at least two of these salts.

In a further preferred embodiment of the invention, the weight ratio of active component a) to active component b) is in the range of 1:50 to 1:1000, particularly preferably in the range of 1:100 to 1:550.

A further object of the invention is medicaments containing the combination of active ingredients according to the invention and optionally further active ingredients and/or excipients.

The medicaments according to the invention are preferably used to control pain, especially to control neuropathic and/or acute pain.

A further object of the invention is also medicament formulations in various administration forms containing the combination of active ingredients according to the invention and optionally further active ingredients and/or excipients.

In a preferred embodiment, the medicament formulations take the form of tablets, lozenges, gum, dragees, capsules, drops, juices, syrups, suppositories, transmucal therapeutic systems, transdermal therapeutic systems, solutions, emulsions, suspensions, easily reconstituted dry preparations, powders or sprays. Particularly preferred medicament formulations are tablets, capsules, drops, solutions, transmucal therapeutic systems or transdermal therapeutic systems.

In a further preferred embodiment, the medicament formulations according to the invention are in multi-particulate form, preferably as micro-tablets, micro-capsules, micro-spheroids, micro-pellets, ion exchange resinates, granulates, active ingredient crystals or pellets, particularly preferably as micro-tablets, granulates or pellets. Pellets in the meaning of the invention also include pellets manufactured by extrusion and/or spheronisation.

The medicament formulations are preferably suitable for oral, intravenous, intramuscular, subcutaneous, intrathecal, epidural, buccal, sublingual, pulmonary, rectal, transdermal, transmucal, nasal or intracerebroventricular administration, medicament formulations for oral, transdermal, transmucal or intravenous administration being particularly preferred.

For oral administration, the preparations preferably take the form of tablets, lozenges, gum, dragees, capsules, granulates, drops, juices and syrups. For buccal administration, a transmucal therapeutic system is preferred. For parenteral, topical and inhalation administration, preferably solutions, suspensions, emulsions, easily reconstituted dry preparations, micro-spheroids, sprays, suppositories or plasters (e.g. transdermal therapeutic systems) are suitable. Particularly preferable are suppositories or solutions for parenteral administration, transdermal therapeutic systems for topical administration and powders or solutions for inhalation administration.

The preparation of the medicament formulations according to the invention may involve, apart from the combination of active ingredients according to the invention, further carrier materials, fillers, solvents, diluents, colorants, flavourings, binders or mixtures of at least two of these materials. The selection of excipients and the quantity thereof depends on how the medicament is to be administered. The appropriate excipients and the quantities thereof for each administration form are known to people skilled in the art. The medicament formulations according to the invention may be manufactured in accordance with the usual methods known to people skilled in the art.

The medicament formulations according to the invention may also contain at least one of the active components a) or b) in retarded (slow release) form.

Retardation of either of the active components is preferably by means of a retarding coating, fixing to an ion exchange resin, by encapsulation in a retarding matrix or by a combination of these different retardations.

Suitable retarding coatings include water-insoluble waxes or polymers such as, for example, acrylic resins, preferably poly(meth)acrylates or water-insoluble celluloses, preferably ethyl cellulose. These materials are known from the prior art, e.g. Bauer, Lehmann, Osterwald, Rothgang “Coated Medicament Forms”, Wissenschaftliche Verlagsgesellschaft mbH Stuttgart, 1988, pp 69 et seq. They are attached as references and thus form part of the disclosure.

In order to adjust the rate of release of either of the active ingredients, the retarding coatings may also contain, in addition to the water-insoluble polymers, non-retarding preferably water-soluble polymers in quantities up to 30% by weight, such as polyvinylpyrrolidone or water-soluble celluloses, preferably hydroxypropylmethylcellulose or hydroxypropylcellulose and/or hydrophilic pore formers, such as sucrose, sodium chloride or mannitol and/or known plasticisers.

Furthermore, the medicament formulation according to the invention may also have further coatings. The coatings may also be such that they dissolve in a pH-dependent manner. In this way, the medicament formulation can pass undissolved through the gastric tract and the combination of active ingredients according to the invention is not released until reaching the intestinal tract. Coatings may also be used to improve the taste.

A further usual procedure for retardation is to fix the active ingredients on ion exchange resins. In order to retard both active component a) and active component b), cation ion exchange resins, preferably polystyrene sulphonate, are used.

For retardation, the combination of active components according to the invention may also be placed in a retarding matrix, preferably uniformly distributed. Physiologically tolerable, hydrophilic materials known to people skilled in the art may be used as matrix materials. Hydrophilic matrix materials are preferably polymers, particularly preferably cellulose ether, cellulose ester and/or acrylic resins. Quite particularly preferable as matrix materials are ethyl cellulose, hydroxypropylmethylcellulose, hydroxypropylcellulose, poly(meth)acrylic acid and/or derivatives thereof, such as salts, amides or esters thereof.

Also preferred are matrix materials comprising hydrophobic materials such as hydrophobic polymers, waxes, fats, long-chain fatty acids, fatty alcohols or corresponding esters or ethers or mixtures thereof. Particularly preferable as hydrophobic materials are mono- or diglycerides of C₁₂-C₃₀ fatty acids and/or C₁₂-C₃₀ fatty alcohols and/or waxes or mixtures thereof.

It is also possible to use mixtures of these hydrophilic and hydrophobic materials as retarding matrix material.

In a further preferred embodiment, the medicament formulation according to the invention contains at least one of active components a) or b) in both retarded and unretarded form. In combination with the active substance which is released immediately, a high initial dose can be achieved for rapid pain alleviation. Slow release from the retarded form then prevents the analgesic effect from subsiding.

The quantity of the combination of active ingredients according to the invention to be administered to patients is known to people skilled in the art from the use of the individual components and varies, for example, in accordance with patient's weight, the type of administration, the indication and the severity of the illness. The quantity to be administered and the release of the combination of active ingredients according to the invention are preferably set so that administration has to take place a maximum of twice. and preferably only once a day.

A further object of the invention is also the use of a combination of active ingredients according to the invention and optionally further active ingredients and/or excipients for the manufacture of a medicament. The combination of active ingredients according to the invention is preferably used to manufacture a medicament to control pain, especially to control neuropathic and/or acute pain.

Surprisingly, the combination of active ingredients according to the invention exhibits a lasting analgesic effect, which reaches a maximum about 15 minutes after administration and is also far improved and far longer lasting after 24 hours in comparison with the administration of each of the two active components a) and b) singly.

This lasting analgesic effect has the advantage that the daily dose of active components a) and b) required for effective pain control can be reduced. This means that the undesirable side effects which usually occur with the administration of active components a) and b) singly, such as respiratory depression, vomiting, dependency, sedation, constipation, the development of tolerance, hallucinogenic effects, impaired coordination, or itching, for example, do not occur or only occur for a considerably shorter period of time and only in a clearly more moderate form.

### Pharmacological research

### Bennett Test on Rats

Research into the analgesic effect of the combination of active ingredients according to the invention and of comparison solutions in the control of neuropathic pain was carried out in accordance with the Bennett model (Bennett and Xie, 1988, Pain 33: 87-107). Male Sprague-Dawley rats (Janvier, France) weighing 140 to 160 grams were used. The rats were first anaesthetised with pentobarbital (50 mg per kg of the rats' bodyweight—Nembutal®, i.p., Sanofi, Wirtschaftsgenossenschaft deutscher Tieraerzte eG, Hanover, Germany). Next, unilateral multiple ligatures were applied to the rats' right main sciatic nerves. For this purpose, the sciatic nerves were exposed at the middle of the thigh and four loose ligatures (softcat®chrom USP 4/0, metric2, Braun Melsungen, Germany) were bound round the sciatic nerves so that the epineural circulation was not cut off. Following this operation, the rats were allowed to recover for a week. The rats developed allodynia against cold, which persisted for at least five weeks.

This allodynia was tested on a metal plate, which was kept at a temperature of 4° C. by means of a water bath. Before administration of the respective solutions, the rats were split into groups of 7 or 8 animals. In order to check the allodynia, the rats were put on the cold metal plate inside a plastic cage. A count was then made over a period of two minutes before administration of a solution with respect to how often the animals violently pulled their injured paws away from the cooled metal plate. The corresponding number of such reactions on the part of the rats is denoted (W_{V}). The corresponding solutions were then administered intravenously and pain measurements were carried out after 15, 30, 45, 60, 120, 180 and 1440 minutes. The corresponding number of reactions on the part of the rats was denoted (W_{N}). The analgesic effect was determined as the decline in the frequency of flinching on the part of the rats (% of the maximum possible antinociceptive effect) in accordance with the following formula:

[(W_{V})−(W_{N})]/(W_{V})×100

Hereinafter, the invention will be illustrated using an example. This example is solely to illustrate the invention and does not restrict the general inventive concept.

### EXAMPLE

In order to investigate the analgesic effect of the combination of active ingredients comprising ketamine and fentanyl, a group of 8 rats were each intravenously given a 0.9% salt solution containing 4.64 mg of ketamine and 0.01 mg of fentanyl per kg of the rats' bodyweight.

### COMPARATIVE EXAMPLE 1

For comparison, a second group of 7 rats were each intravenously given a 0.9% salt solution containing only 4.64 mg of ketamine per kg of the rats' bodyweight.

### COMPARATIVE EXAMPLE 2

For comparison, a third group of 7 rats were each intravenously given a 0.9% salt solution containing only 0.01 mg of fentanyl per kg of the rats' bodyweight.

The results of these tests are shown in FIG. 1.

FIG. 1 shows that the comparison solution in accordance with comparative example 1, which contains only ketamine, exhibits a good analgesic effect about 15 minutes after administration, which lasts for a period of about 3 hours and then fades away.

The comparison solution in accordance with comparative example 2, which contains only fentanyl, exhibits a good analgesic effect for a period of about 15 minutes after administration, which then, however, fades away very rapidly. One hour after administration, fentanyl alone exhibits almost no analgesic effect any longer.

As can be seen from FIG. 1, the administration of the solution of the combination of active ingredients comprising ketamine and fentanyl has an analgesic effect which reaches its maximum about 15 minutes after administration and the analgesic effect of which is clearly improved over a period of about 45 minutes after administration in comparison with the single administration of ketamine or fentanyl. Even 1440 minutes, i.e. 24 hours after administration, the solution of the combination of active ingredients according to the invention still exhibits a clearly expressed analgesic effect, while the comparison solutions in accordance with comparative examples 1 and 2 no longer exhibit any analgesic effect after this period of time.

1. Combination of active ingredients containing
a) at least one opioid compound with a fentanyl-type structure and/or one of its enantiomers and/or one of its diastereomers and/or a corresponding physiologically tolerable salt and
b) ketamine and/or at least one of its physiologically tolerable salts,
characterised in that the weight ratio of active component a) to active component b) is in the range of 1:20 to 1:1500.

2. Combination of active ingredients according to claim 1, characterised in that at least one compound of general formula I 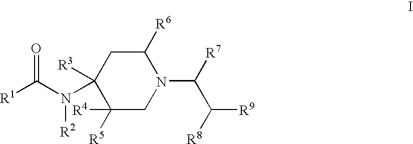
where
group R¹ stands for a C₁₋₃-alkyl, a C₁₋₃-alkoxymethyl or a 2-furanyl group,
group R² stands for a phenyl group or a phenyl group optionally substituted with fluorine in the ortho-position or a 2-pyrazinyl group,
group R³ stands for H, a C₁₋₃-alkoxymethyl, a C₁₋₃-alkoxycarbonyl or a phenyl group,
groups R⁴ and R⁵, the same or different, each stand for H, OH or a C₁₋₃-alkyl group,
groups R⁶ and R⁷, the same or different, each stand for H or a C₁₋₃-alkyl group, group R⁸ stands for H or OH and
group R⁹ stands for a phenyl, a 2-thienyl, a C₁₋₃-alkoxycarbonyl or a 1-ethyl-1,4-dihydro-tetrazol-5-one group,
and/or one of its enantiomers and/or one of its diastereomers and/or a corresponding physiologically tolerable salt is present as the opioid compound with a fentanyl-type structure.

3. Combination of active ingredients according to claims 1 or 2, characterised in that fentanyl, alfentanil, brifentanil, carfentanil, fenaridine, fentatienil, lofentanil, ocfentanil, mefentanil, mirfentanil, remifentanil, sufentanil, trefentanil and/or one of the enantiomers thereof and/or one of the diastereomers thereof and/or at least one corresponding physiologically tolerable salt or a mixture of at least two of the above mentioned compounds is present as the opioid compound with a fentanyl-type structure.

4. Combination of active ingredients according to one of claims 1 to 3, characterised in that the weight ratio of active component a) to active component b) is in the range of 1:125 to 1:1000, preferably in the range of 1:350 to 1:550.

5. Combination of active ingredients according to one of claims 1 to 4, characterised in that hydrochloride, hydrobromide, sulphate, sulphonate, phosphate, tartrate, formiate, acetate, propionate, benzoate, oxalate, succinate, citrate, glutamate, embonate, fumarate, aspartate, glutarate, stearate, butyrate, malonate, lactate, mesylate or a mixture of at least two of these salts is present as a physiologically tolerable salt of the opioid compound with a fentanyl-type structure and/or its enantiomers and/or its diastereomers.

6. Combination of active ingredients according to one of claims 1 to 5, characterised in that hydrochloride, hydrobromide, sulphate, sulphonate, phosphate, tartrate, formiate, acetate, propionate, benzoate, oxalate, succinate, citrate, glutamate, embonate, fumarate, aspartate, glutarate, stearate, butyrate, malonate, lactate, mesylate or a mixture of at least two of these salts is present as a physiologically tolerable salt of ketamine.

7. Medicament containing a combination of active ingredients according to one of claims 1 to 6 and optionally further active ingredients and/or excipients.

8. Medicament according to claim 7 for controlling pain.

9. Medicament according to claim 8 for controlling neuropathic pain.

10. Medicament according to claim 8 for controlling acute pain.

11. Medicament formulation containing a combination of active ingredients according to one of claims 1 to 6 and optionally further active ingredients and/or excipients.

12. Medicament formulation according to claim 11, characterised in that it takes the form of tablets, lozenges, gum, dragees, transdermal therapeutic systems, transmucal therapeutic systems, capsules, suppositories, drops or of juice, syrup, solution, emulsion, suspension, easily reconstituted dry preparation, powder or spray, preferably in the form of tablets, capsules, drops or solution.

13. Medicament formulation according to claim 11, characterised in that it takes a multi-particulate form, preferably in the form of micro-tablets, micro-capsules, micro-spheroids, ion exchange resinates, granulates, active ingredient crystals or pellets, particularly preferably in the form of micro-tablets, granulates or pellets.

14. Medicament formulation according to one of claims 11 to 13 for oral, intravenous, intramuscular, subcutaneous, intrathecal, epidural, buccal, sublingual, rectal, pulmonary, transdermal, transmucal, nasal or intracerebroventricular, preferably for oral, transdermal, transmucal or intravenous administration.

15. Medicament formulation according to one of claims 11 to 14, characterised in that at least one of active components a) or b) is present in retarded form.

16. Medicament formulation according to claim 15, characterised in that retardation is achieved by means of a retarding coating, fixing to an ion exchange resin, by encapsulation in a retarding matrix or by a combination of these different retardations

17. Medicament formulation according to claim 16, characterised in that the coating is based on a water-insoluble polymer or wax.

18. Medicament formulation according to claim 17, characterised in that a polyacrylic resin or cellulose derivative, preferably alkyl cellulose, is used as the water-insoluble polymer.

19. Medicament formulation according to claim 18, characterised in that ethylcellulose and/or a poly(meth)acrylate is used as the polymer.

20. Medicament formulation according to claim 16, characterised in that the matrix contains hydrophilic matrix materials, preferably polymers, particularly preferably cellulose ether, cellulose ester and/or acrylic resins, quite particularly preferably ethyl cellulose, hydroxypropylmethylcellulose, hydroxypropylcellulose, hydroxymethylcellulose, poly(meth)acrylic acid and/or salts thereof and/or amides thereof and/or esters thereof.

21. Medicament formulation according to claim 16 or 20, characterised in that the matrix contains hydrophobic matrix materials, preferably polymers, waxes, fats, long-chain fatty acids, fatty alcohols or corresponding esters or ethers or mixtures thereof, particularly preferably mono- and diglycerides of C₁₂-C₃₀ fatty acids and/or C₁₂-C₃₀ fatty alcohols and/or waxes or mixtures thereof.

22. Medicament formulation according to claim 16, characterised in that polystyrene sulphonates are used as cation exchange resins.

23. Medicament formulation according to one of claims 15 to 22, characterised in that at least one of active components a) or b) is present in unretarded form as well as retarded form.

24. Use of a combination of active ingredients according to one of claims 1 to 6 and optionally further active ingredients and/or excipients for the manufacture of a medicament.

25. Use according to claim 24 for the manufacture of a medicament for controlling pain.

26. Use according to claim 25 for the manufacture of a medicament for controlling neuropathic pain.

27. Use according to claim 25 for the manufacture of a medicament for controlling acute pain.
